(19) United States
(12) Patent Application Publication
(10) Pub. No.: US 2023/0218207 A1
(43) Pub. Date: Jul. 13, 2023

(54) SUBSTANCE CONCENTRATION MEASURING DEVICE, SUBSTANCE CONCENTRATION MEASURING METHOD AND HUMAN CONDITION MONITORING SYSTEM

(71) Applicant: PixArt Imaging Inc., Hsin-Chu City (TW)

(72) Inventors: Chih-Hao Wang, Hsin-Chu City (TW); Chien-Yi Kao, Hsin-Chu City (TW); Yang-Ming Chou, Hsin-Chu City (TW); Shih-Jen Lu, Hsin-Chu City (TW); Hsin-Yi Lin, Hsin-Chu City (TW)

(73) Assignee: PixArt Imaging Inc., Hsin-Chu City (TW)

(21) Appl. No.: 17/574,574

(22) Filed: Jan. 13, 2022

(51) Int. Cl. A61B 5/145; A61B 5/297; A61B 5/398; A61B 5/00; A61B 5/315; A61B 5/18

(52) U.S. Cl. CPC A61B 5/14546; A61B 5/297; A61B 5/398; A61B 5/4845; A61B 5/315; A61B 5/6802; A61B 5/18

(57) ABSTRACT

A substance concentration measuring device, for measuring a substance concentration for a target substance in blood of a subject, comprising: a processing circuit; and a plurality of electrodes. At least two of the electrodes touches the subject when the subject wears the substance concentration measuring device, thereby the processing circuit can acquire at least one physiological signal caused by an eye of the subject. The processing circuit computes a voltage value or a voltage difference of the physiological signal, and computes the substance concentration according to the voltage value or the voltage difference. A human condition monitoring system comprising the substance concentration measuring device is also disclosed.

### BACKGROUND OF THE INVENTION

### 1. Field of the Invention

The present invention relates to a substance concentration measuring device, a substance concentration measuring method and a human condition monitoring system, and particularly relates to a substance concentration measuring device, a substance concentration measuring method and a human condition monitoring system which can computes the substance concentration according to a physiological signal caused by an eye of a subject.

### 2. Description of the Prior Art

Under certain circumstances, it may need to measure substance concentration for a target substance in blood of a subject. For example, measuring alcohol concentration or drug concentration in blood for a driver, a machine operator, or a medical personnel. However, in conventional methods, many subjects may need to use the same tester while being tested.

For example, the drivers always blow the same blower for alcohol measurement. Such method is not good for public health, especially when infectious diseases are prevalent. Also, the result of such test may be unstable since it only reveals breathing alcohol level, whose scale is much smaller than blood alcohol level.

### SUMMARY OF THE INVENTION

One objective of the present invention is to provide a substance concentration measuring device for measuring a substance concentration for a target substance in blood of a subject according to at least one physiological signal caused by an eye of the subject.

Another objective of the present invention is to provide human condition monitoring system for measuring the substance concentration the for performing an operation according to the substance concentration.

Still objective of the present invention is to provide a substance concentration measuring method for measuring a substance concentration for a target substance in blood of a subject according to at least one physiological signal caused by an eye of the subject.

One embodiment of the present invention discloses a substance concentration measuring device, for measuring a substance concentration for a target substance in blood of a subject, comprising: a processing circuit; and a plurality of electrodes. At least two of the electrodes touches the subject when the subject wears the substance concentration measuring device, thereby the processing circuit can acquire at least one physiological signal caused by an eye of the subject. The processing circuit computes a voltage value or a voltage difference of the physiological signal, and computes the substance concentration according to the voltage value or the voltage difference.

Another embodiment of the present invention discloses a human condition monitoring system, for monitoring a condition of a subject, comprising: a control circuit and a substance concentration measuring device. The substance concentration measuring device is for measuring a substance concentration for a target substance in blood of the subject, comprises: a processing circuit; and a plurality of electrodes. At least two of the electrodes touches the subject when the subject wears the substance concentration measuring device, thereby the processing circuit can acquire at least one physiological signal caused by an eye of the subject. The processing circuit computes a voltage value or a voltage difference of the physiological signal, and computes the substance concentration according to the voltage value or the voltage difference. The control circuit controls the human condition monitoring system to perform a specific operation according to a relation between the substance concentration and a threshold.

Still another embodiment of the present invention discloses a substance concentration measuring method, applied to a substance concentration measuring device comprising a plurality of electrodes, comprising: (a) acquiring at least one physiological signal caused by an eye of a subject via at least two of the electrodes which touch the subject; and (b) computing a voltage value and a voltage difference of the physiological signals, and computes the substance concentration according to the voltage value and the voltage difference.

In view of above-mentioned embodiments, the substance concentration can be acquired by physiological signal caused by an eye rather than by breath of the subject. Accordingly, public health can be better and the measurement result can be more stable.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram illustrating a substance concentration measuring device according to one embodiment of the present invention.

FIG. 2 is a schematic diagram illustrating locations of the electrodes according to one embodiment of the present invention.

FIG. 3 is a schematic diagram illustrating voltage variation when the eye is rotating and the eye is not rotating.

FIG. 4-FIG. 6 are different types of the substance concentration measuring devices, according to different embodiments of the present invention.

FIG. 7 is a flow chart illustrating a substance concentration measuring method according to one embodiment of the present invention.

### DETAILED DESCRIPTION

Several embodiments are provided in following descriptions to explain the concept of the present invention. Each component in following descriptions can be implemented by hardware (e.g. a device or a circuit) or hardware with software (e.g. a program installed to a processor). Besides, the method in following descriptions can be executed by programs stored in a non-transitory computer readable recording medium such as a hard disk, an optical disc or a memory. Additionally, the term “first”, “second”, “third” in following descriptions are only for the purpose of distinguishing different one elements, and do not mean the sequence of the elements. For example, a first device and a second device only mean these devices can have the same structure but are different devices.

FIG. 1 is a block diagram illustrating a substance concentration measuring device 100 according to one embodiment of the present invention, which is for measuring a substance concentration for a target substance in blood of a subject. As illustrated in FIG. 1, the substance concentration measuring device 100 comprises a processing circuit 101 and a plurality of electrodes EC1, EC2, EC3 . . . (only three of the electrodes are symbolized). Please note, in following embodiments, the target substance is alcohol and the substance concentration is alcohol concentration. However, the target substance can be any other substance.

In one embodiment, the substance concentration measuring device 100 is provided in a wearable device. Also, at least two of the electrodes EC1, EC2, EC3 touches the subject when the subject wears the substance concentration measuring device 100, thereby the processing circuit 101 can acquire at least one physiological signal caused by an eye of the subject. However, the substance concentration measuring device 100 is not limited to be provided in a wearable device, it can be implemented by other types of devices. The processing circuit 101 computes a voltage value or a voltage difference of the physiological signal, and computes the substance concentration according to the voltage value or the voltage difference.

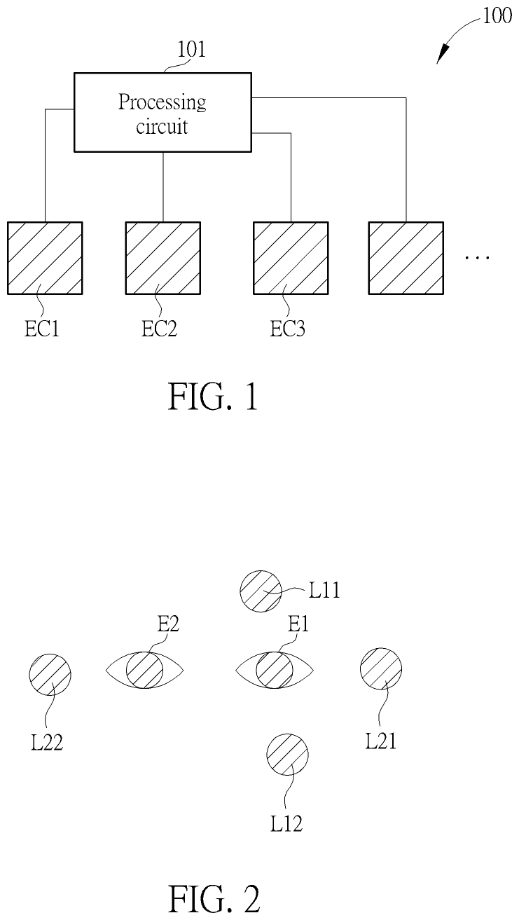

In one embodiment, the physiological signal is an EOG (Electrooculogram) signal. For measuring the EOG signal, at least two of the electrodes are provided near the eyes of the subject. In one embodiment, the at least two electrodes touch symmetrical locations of the eyes. FIG. 2 is a schematic diagram illustrating locations of the electrodes according to one embodiment of the present invention. As illustrated in FIG. 2, two electrodes can be respectively provided at the locations L11, L12, which are respectively close to an upper eyelid and a lower eyelid of the eye E1. Alternatively, the two electrodes can be provided at the locations L21, L22, which are respectively close to a left eye corner of the eye E2 and a right eye corner of the eye E1. Besides the electrodes touching the subject, a reference electrode can be provided as a ground. Such reference electrode can touch another portion of the same subject or touch any other reference surface such as a desk surface. Please note, the locations of the electrodes are not limited to the example illustrated in FIG. 2.

Electrooculography (EOG) is a technique for measuring the corneo retinal standing potential that exists between the front and the back of the human eye, as known by persons skilled in the art. The EOG signal can be applied for measuring eye movements. FIG. 3 is a schematic diagram illustrating voltage variation when the eye is rotating and the eye is not rotating. As illustrated in FIG. 3, when the eye 300 has no rotation, the EOG signal ES has a constant voltage value shown by a solid line. Also, if the eye 300 has right rotation or left rotation, the voltage value of the EOG signal ES varies as shown by two solid lines, thus voltage differences of the EOG signal ES can be detected.

Alcohol concentration in the blood may affect the voltage value or the voltage difference of the EOG signal, as shown in the dotted lines in FIG. 3. Please note, the dotted lines in FIG. 3 are only examples for explaining. It does not mean the alcohol must cause the same variation for subjects under different situations. Details of the alcohol affect for EOG signal can be found in various published documents, such as the paper “The human electro-oculogram: interaction of light and alcohol, G B Arden, J E Wolf, August 2000”.

Therefore, the processing circuit 101 computes the substance concentration according to the voltage value which is generated when the eye is not rotating or the voltage difference when the eye is rotating. In one embodiment, a reference voltage value is acquired by collecting and averaging the voltage values of EOG signals for a predetermined number of reference subjects which do not drink, eat or inject any material with alcohol and do not make eye rotation. Also, the voltage value of the EOG signal of a subject is compared with the reference voltage value, to determine an alcohol concentration of the subject. In one embodiment, the larger the difference between the voltage value and the reference voltage value, the larger the alcohol concentration is. In one embodiment, a mapping table of the alcohol concentration and the difference can be acquired via a plurality experiments. In such case, the real alcohol concentration can be acquired based on the difference and the mapping table.

Similarly, in one embodiment, a reference voltage difference is acquired by collecting and averaging the voltage differences of EOG signals for a predetermined number of reference subjects which do not drink, eat or inject any material with alcohol but makes eye rotation in at least one specific manner. In such case, the voltage difference of the EOG signal of a subject is compared with the reference voltage difference, to determine an alcohol concentration of the subject. In one embodiment, the larger the difference between the voltage difference and the reference voltage difference, the larger the alcohol concentration is. Also, the real alcohol concentration can be acquired based on the difference between the voltage difference and the reference voltage difference and the mapping table.

The reference voltage value can be acquired by any other method. In one embodiment, a reference voltage value is acquired by collecting and averaging the voltage values of EOG signals for a single subject which does not drink, eat or inject any material with alcohol and do not make eye rotation, for a predetermined numbers at different time. Also, the voltage value of the EOG signal of the same subject is compared with the reference voltage value, to determine an alcohol concentration of the subject, after the reference voltage value is established and when the alcohol concentration of the same subject needs to be measured.

Similarly, in one embodiment, a reference voltage difference is acquired by collecting and averaging the voltage differences of EOG signals for a single subject which does not drink, eat or inject any material with alcohol but makes eye rotation in at least one specific manner, for a predetermined numbers at different time. Also, the voltage difference of the EOG signal of the same subject is compared with the reference voltage reference, to determine an alcohol concentration of the subject, after the reference voltage difference is established.

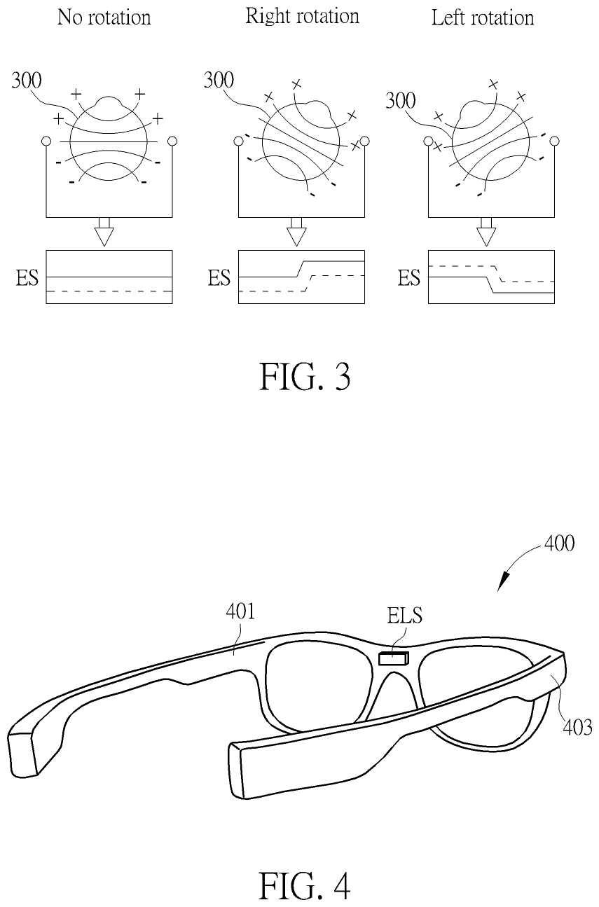

As above-mentioned, the substance concentration measuring device 100 can be provided in a wearable device but can be provided to any other type of device. FIG. 4-FIG. 6 are different types of the substance concentration measuring devices. In the embodiment of FIG. 4, the substance concentration measuring device 100 is provided in a spectacle frame 400, and electrodes ELS are provided at a middle portion of the spectacle frame 400. By this way, the electrode ELS touch a portion between eyes of a subject when the subject wears the spectacle frame 400. Also, as above-mentioned, the electrode can touch the temple of the subject to sense physiological signals. In such case, the electrode can be provided at the location 401 or the location 403 in FIG. 4, to touch the temple of the subject. Additionally, in the embodiment of FIG. 4, the processing circuit 101 in FIG. 1 can be provided inside the spectacle frame 400, but can be provided outside the spectacle frame 400 as well.

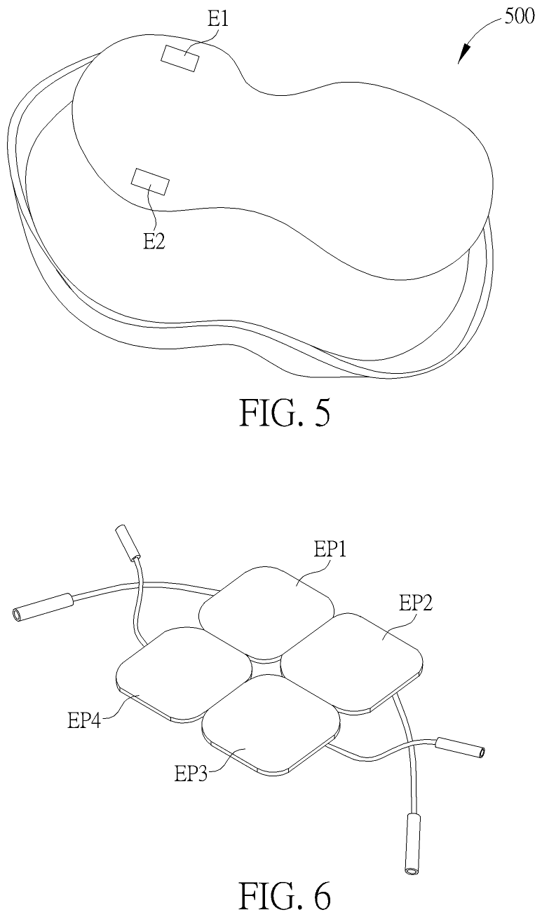

The wearable device can be a device in any other form besides the spectacle frame 400 in FIG. 4. As illustrated in FIG. 5, the substance concentration measuring device 100 is provided in a blindfold 500. In such embodiment, the electrodes EL1, EL2 are provided as illustrated in FIG. 5. By this way, the electrodes EL1, EL2 touches the locations L11, L12 illustrated in FIG. 2 when the subject wears the blindfold 500. Additionally, in the embodiment of FIG. 5, the processing circuit 101 in FIG. 1 can be provided inside the blindfold 500, but can be provided outside the blindfold 500 as well.

As above-mentioned, the substance concentration measuring device 100 is not limited to be implemented by a wearable device. As illustrated in the embodiment of FIG. 6, each of the electrode patches EP1, EP2, EP3, and EP4 comprises at least one electrode. The electrode patches EP1, EP2, EP3, and EP4 comprise sticky material thus can be pasted wherever you want. Therefore, at least one of the electrode patches EP1, EP2, EP3, and EP4 can be pasted to suitable locations of an subject for EOG signal measuring, such as the locations illustrated in FIG. 2. In such case, the processing circuit 101 can be provided in a control device which is connected to the electrode patches EP1, EP2, EP3, and EP4, to perform above-mentioned embodiments.

In one embodiment, the substance concentration measuring device 100 is applied to a human condition monitoring system. Besides the substance concentration measuring device 100, the human condition monitoring system also comprises a control circuit. After the alcohol concentration is acquired, the control circuit controls the human condition monitoring system to perform a specific operation according to a relation between the alcohol concentration and a threshold. For example, a driver can wear the human condition monitoring system for measuring the alcohol concentration before driving. The human condition monitoring system can generate warning messages, notifying the supervisory unit or even locking down the vehicle which the driver wants to drive, if the alcohol concentration is too high (i.e., higher than the threshold). Please note, the control circuit of the human condition monitoring system and the processing circuit of the substance concentration measuring device 100 can be integrated to a single circuit or a single chip.

As above-mentioned, the target substance can be any other substance besides the alcohol. Therefore, in such case, the human condition monitoring system can generate warning messages, notifying the supervisory unit or even locking down the vehicle which the driver wants to drive if the substance concentration is too low (i.e., lower than the threshold) thus may causes the driver to become drowsy or unconscious. The substance concentration mentioned here can be endocrine substance.

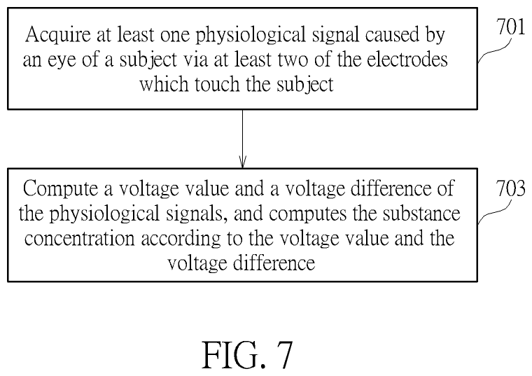

FIG. 7 is a flow chart illustrating a substance concentration measuring method according to one embodiment of the present invention.

Step 701

Acquire at least one physiological signal caused by an eye of a subject via at least two of the electrodes which touch the subject.

Step 703

Compute a voltage value and a voltage difference of the physiological signals, and computes the substance concentration according to the voltage value or the voltage difference.

In view of above-mentioned embodiments, the substance concentration can be acquired by physiological signal caused by an eye rather than by breath of the subject. Accordingly, public health can be better and the measurement result can be more stable.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A substance concentration measuring device, for measuring a substance concentration for a target substance in blood of a subject, comprising:
a processing circuit; and
a plurality of electrodes;
wherein at least two of the electrodes touches the subject when the subject wears the substance concentration measuring device, thereby the processing circuit can acquire at least one physiological signal caused by an eye of the subject;
wherein the processing circuit computes a voltage value or a voltage difference of the physiological signal, and computes the substance concentration according to the voltage value or the voltage difference.

2. The substance concentration measuring device of claim 1, wherein the physiological signal is an EOG (Electrooculogram) signals.

3. The substance concentration measuring device of claim 2, wherein the target substance is alcohol.

4. The substance concentration measuring device of claim 1, wherein the processing circuit computes the voltage value according the physiological signal which is generated when the eye is not rotating.

5. The substance concentration measuring device of claim 1, wherein the processing circuit computes the voltage difference according the physiological signals which are generated when the eye is rotating.

6. The substance concentration measuring device of claim 1, wherein the at least two electrodes touch symmetrical locations of the eyes.

7. The substance concentration measuring device of claim 1, wherein one of the at least two electrodes touches a temple of the subject.

8. A human condition monitoring system, for monitoring a condition of a subject, comprising:
a control circuit; and
a substance concentration measuring device, for measuring a substance concentration for a target substance in blood of the subject, comprising:
a processing circuit; and
a plurality of electrodes;
wherein at least two of the electrodes touches the subject when the subject wears the substance concentration measuring device, thereby the processing circuit can acquire at least one physiological signal caused by an eye of the subject;
wherein the processing circuit computes a voltage value or a voltage difference of the physiological signal, and computes the substance concentration according to the voltage value or the voltage difference;
wherein the control circuit controls the human condition monitoring system to perform a specific operation according to a relation between the substance concentration and a threshold.

9. The human condition monitoring system of claim 8, wherein the physiological signal is an EOG (Electrooculogram) signals.

10. The human condition monitoring system of claim 9, wherein the target substance is alcohol.

11. The human condition monitoring system of claim 8, wherein the processing circuit computes the voltage value according the physiological signals which is generated when the eye is not rotating.

12. The human condition monitoring system of claim 8, wherein the processing circuit computes the voltage difference according the physiological signals which are generated when the eye is rotating.

13. The human condition monitoring system of claim 8, wherein the at least two electrodes touch symmetrical locations of the eyes.

14. The human condition monitoring system of claim 8, wherein one of the at least two electrodes touches a temple of the subject.

15. A substance concentration measuring method, applied to a substance concentration measuring device comprising a plurality of electrodes, comprising:
(a) acquiring at least one physiological signal caused by an eye of a subject via at least two of the electrodes which touch the subject; and
(b) computing a voltage value and a voltage difference of the physiological signals, and computes the substance concentration according to the voltage value and the voltage difference.

16. The substance concentration measuring method of claim 15, wherein the physiological signals are EOG (Electrooculogram) signals.

17. The substance concentration measuring method of claim 16, wherein the target substance is alcohol.

18. The substance concentration measuring method of claim 15, wherein the step (b) computes the voltage difference according the physiological signals which are generated when the eye is not rotating.

19. The substance concentration measuring method of claim 15, wherein the step (b) computes the voltage difference according the physiological signals which are generated when the eye is rotating.

20. The substance concentration measuring method of claim 15, wherein the at least two electrodes touch symmetrical locations of the eyes.

21. The substance concentration measuring method of claim 15, wherein one of the at least two electrodes touches a temple of the subject.
